(19) United States
(12) Patent Application Publication
(10) Pub. No.: US 2023/0044608 A1
(43) Pub. Date: Feb. 9, 2023

(54) Compounds for treatment of eye diseases

(71) Applicants: Aarhus Universitet, Aarhus C (DK); Region Midtjylland, Viborg (DK)

(72) Inventors: Anders Nykjær, Risskov (DK); Anne Louise Askou, Hinnerup (DK); Thomas Juhl Corydon, Risskov (DK); Toke Bek, Risskov (DK)

(21) Appl. No.: 17/783,143

(22) PCT Filed: Dec. 10, 2020
(86) PCT No.: PCT/EP2020/085539

§ 371 Date: Jun. 7, 2022

(30) Foreign Application Priority Data
Dec. 10, 2019 (EP) 19214731.2

(51) Int. Cl. C07K 16/28; A61K 39/395; A61P 27/02

(52) U.S. Cl. CPC C07K 16/28; A61K 39/395; A61P 27/02; A61K 2039/505

(57) ABSTRACT

The present invention concerns an anti-sortilin antibody or an antigen binding fragment thereof, for use in the treatment or prevention of a disease of the eye, in particular diseases or disorders of the retina, the choroid and/or the optic nerve.

### TECHNICAL FIELD

The present invention relates to compounds binding to the receptor sortilin, for use in the treatment or prevention of diseases or disorders of the retina, the choroid and/or the optic nerve.

### BACKGROUND

Diabetic retinopathy (DR) is one of the most common complications of diabetes mellitus (DM). It is a specific microvascular complication and the leading cause of vision loss in the general population in the Western world (Wong 2016). The estimated number of adults with DM worldwide is 422 million persons (NCD Risk Factor Collaboration 2016). The risk of developing DR increases with increasing diabetes duration and the prevalence of DR is approximately 35% among the patients (Yau 2012). The exact pathogenesis of DR is currently unknown, but involves microvascular changes leading to capillary occlusion and ischaemia in the retinal periphery and hyperperfusion and hyperpermeability in the macular area (Wong 2016). Vascular endothelial growth factor (VEGF) released from the pathological retinal tissue is an important mediator for the initiation of these changes, and current treatment involves a reduction in the synthesis of this compound by retinal photocoagulation and binding of the compound by anti-VEGF antibodies or agents that inhibit the activity of VEGF. However, these treatment modalities only treat symptoms and not the underlying disease (Antonetti 2012). More recent findings further suggest reactive gliosis and neurodegeneration (Pincello Netto 2018, De Clerck 2018, Oshitari 2009) to play a significant role in this complex diabetic complication, where a special emphasis has been placed on the pathophysiology of the neurovascular unit. These findings have led to studies that explore anti-inflammatory and neuroprotective treatment strategies.

Besides DR, vaso-occlusion, ischemia, and neurodegeneration with loss of tissue in the inner retina are present in a range of retinal vascular pathologies as well as optic nerve diseases such as retinal vein occlusion, primary open-angle glaucoma, and age-related macular degeneration.

Sortilin (Petersen 1997) is a neurotensin receptor belonging to the Vps10p domain family (Jacobsen 2001, Hermey 2003). Studies in cell lines, in animal models, and in patient tissue have confirmed the causal role of deregulation of sortilin expression in numerous disease processes (Evans 2011, Jansen 2007, Carlo 2013, Gustafsen 2013 Kjolby 2010), where several cellular mechanisms have been revealed. One mechanism, apoptotic signalling in neurons, has been thoroughly investigated (Jansen 2007, Nykjaer 2004). Nerve growth factor (NGF) belongs to the family of neurotrophins that are essential factors to stimulate neuronal development, integrity and functionality (Skeldal 2011). However, neurotrophins (NGF, BDNF, NT3, NT4) are commonly secreted as pro-neurotrophins (proNGF, proBDNF, proNT3), which are, opposed to their mature counterparts, proapoptotic. Whereas NGF targets the tyrosine receptor kinase A (TrkA) complexed with the p75 neurotrophin receptor (p75^{NTR})for trophic signalling, pro-nerve growth factor (proNGF) engages a receptor complex comprising sortilin (or SorCS2) and the p75^{NTR} for induction of apoptosis (Nykjaer 2004, Glerup 2014). Similar systems operate for proBDNF and proNT3, the only difference being that the tyrosine receptor kinases are TrkB and TrkC, respectively. A vast number of studies have demonstrated that proNGF-induced cell death signalling is critical to several neurodegenerative diseases (Evans 2011, Carlo 2013, Gustafsen 2013, Finan 2011, Yang 2011) and also to DM as a neurodegenerative disorder secondary to DM (Mohamed 2015). Moreover, DM has been shown to cause imbalance of proNGF and NGF in the human retina, in favour of the preform (Ali 2011).

The role of sortilin and sortilin inhibition has to date not been elucidated in disease processes of the retina induced by diabetes and/or pathological amounts of proNGF.

Conclusively, there is an unmet need to prevent the development of DR in patients with DM as well as to identify treatment modalities which do not only treat symptoms and but the underlying disease.

### SUMMARY

The inventors of the present invention have found that inhibition of the receptor sortilin by anti-sortilin antibodies protects retinal neurons from degeneration.

Thus, in one aspect the present invention concerns a composition comprising or consisting of an anti-sortilin antibody or an antigen binding fragment thereof, for use in the treatment or prevention of a disease or disorder of the retina, the choroid and/or the optic nerve.

In a further aspect, the present invention relates to a composition comprising or consisting of an anti-sortilin antibody or an antigen binding fragment thereof, for the manufacture of a medicament for the treatment or prevention of a disease of the retina, the choroid and/or the optic nerve.

In a another aspect, the present invention relates to a method of treatment of a disease of the retina, the choroid and/or the optic nerve, the method comprising administering to an individual in need thereof a therapeutically effective amount of an anti-sortilin antibody or an antigen binding fragment thereof.

In a further aspect, the present invention relates to a composition comprising or consisting of an anti-sortilin antibody or an antigen binding fragment thereof, for use in the diagnosis of a disease of the retina, the choroid and/or the optic nerve.

In a further aspect, the present invention relates to a method for detecting a disease of the retina, the choroid and/or the optic nerve by using an anti-sortilin antibody or an antigen binding fragment thereof, said method comprising analysing in a sample obtained from a mammalian retina, choroid or optic nerve, the presence or absence of an antigen comprising a sortilin polypeptide, wherein the presence of the antigen is indicative of a disease of the retina, the choroid and/or the optic nerve.

### DESCRIPTION OF DRAWINGS

FIG. 1: Sortilin and p75NTR overexpression and co-localization in diabetic retinopathy. (A) Paraffin-sections labeled with sortilin (1st column) and p75^{NTR} (2nd column) and co-localization (merge and arrows) in patients diagnosed with diabetic retinopathy (DR; upper row) compared to a healthy control (lower low). (B) Mean fluorescent signal intensity of sortilin (black rectangles) and p75^{NTR} (grey circles) in different retinal layers (nerve fiber layer (NFL), ganglion cell layer (GCL), inner plexiform layer (IPL), inner nuclear layer (INL), outer plexiform layer (OPL), outer nuclear layer (ONL) outer limiting membrane (OLM) and inner and outer segments of the photoreceptors (PR I/O) in the diabetic group (solid lines) and the control group (dotted lines). The different retinal layers are annotated in FIG. 1A. DR: diabetic retinopathy.

FIG. 2: Sortilin is localized in retinal Muller cells in the human retina. Paraffin-sections labeled with sortilin (1^{st} column) and a Muller cell marker (2^{nd} column) show co-localization (merge and arrows), both in patients diagnosed with diabetic retinopathy (upper row) and healthy controls (lower row). GFAP is found abundantly in a diabetic retina, but is not detectable in a healthy retina. Hence, GS is used as Muller cell marker in the control retinas. DR: diabetic retinopathy GFAP: glial fibrillary acidic protein; GS: gluthamine synthetase.

FIG. 3: Sortilin and p75^{NTR} co-localize in the diabetic murine retina. Paraffin-sections labeled with sortilin (1^{st} column) and p75^{NTR} (2^{nd} column) show co-localization almost exclusively in diabetic murine retinas (merge and arrows). The areas delineated by the dotted squares are magnified in the 4^{th} column. DM: diabetes mellitus.

FIG. 4: Sortilin is localized in retinal Müller cells in the retina of diabetic mice. Paraffin-sections labeled with sortilin (1^{st} column) and a Muller cell marker (2^{nd} column) show co-localization primarily in diabetic murine retinas (merge and arrows). DM: diabetes mellitus; GS: gluthamine synthetase.

FIG. 5: Sortilin and p75^{NTR} levels are increased in the diabetic murine retina. (A) Images of Western blots for quantification of protein levels of sortilin and p75^{NTR} in the diabetic murine retina. Antibodies against GAPDH were utilized for the loading control. The quantified amount of sortilin relative to GAPDH (sortilin/GAPDH) and p75^{NTR}/GAPDH in diabetic mice (DM; black bars) compared to non-diabetic mice (non-DM; white bars) are shown on the right. The mean level of sortilin and p75^{NTR} protein in the non-diabetic retinas was set to 100. p values were p=0.02 (sortilin) and p=0.0002 (p75^{NTR}). *Statistically significant. Error bars indicate STD. (B) Assessment of sortilin and p75^{NTR} mRNA expression in the retina of diabetic mice. RNA was purified from total neuroretina of 4 diabetic mice (DM; black rhombuses) and 4 non-diabetic mice (non-DM; white rhombuses). The sortilin and p75^{NTR} expression was related to the expression of the HPRT housekeeping gene. The mean expression of sortilin and p75^{NTR} in the non-diabetic mouse retinas was set to 100. p values were p=0.01 (sortilin) and p=0.004 (p75^{NTR}). *Statistically significant. DM: diabetes mellitus; GAPDH; Glyceraldehyde 3-phosphate dehydrogenase; HPRT, hypoxanthine phosphoribosyltransferase.

FIG. 6: The streptozotocin-induced mouse model of type 1 diabetes mellitus. 8-weeks-old C57BI6/J male mice were given 5 consecutive daily intraperitoneal injections of low amounts of streptozotocin (STZ). Blood glucose levels were measured 1 week (T₀) after the first STZ injection (T₋₁) and all mice with post-prandial blood glucose levels>15 mM were included in the study. Mice were anesthetized 2.5 weeks after T₀ (T_{2.5}), optical coherence tomography (OCT) images of the retinas were obtained from all mice and subsequently, 20 diabetic mice were injected intravitreal (IVT) with 2 μg anti-sortilin polyclonal antibody in the left eye and an equal volume PBS in the right eye. Retinal images were obtained by OCT 4 (T₄), 6 (T₆) and 8 (T₈) weeks after T₀. At T₈, mice were sacrificed and retinas harvested for subsequent analysis. DM: diabetes mellitus; pAb: polyclonal antibody; STZ: streptozotocin.

FIG. 7: Anti-sortilin protects the inner retina from degeneration after onset of diabetes. OCT images of diabetic mice injected with anti-sortilin antibody (middle column) compared to diabetic mice injected with PBS (right column) and non-diabetic mice (left column) after 4, 6, and 8 weeks of diabetes (see FIG. 6 for experimental setup). Retinal thickness of the NGI (Nerve fiber layer, Ganglion cell layer and Inner plexiform layer; white bar) is measured in pm and shown in the graph 2.5, 4, 6, and 8 weeks after the onset of diabetes. Mean values±STD are shown for non-diabetic control mice with black squares, diabetic mice injected with PBS with grey triangles and diabetic mice injected with anti-sortilin with white circles. *Statistically significant. DM: diabetes mellitus.

FIG. 8: Administration of anti-sortilin antibody protects the retinal ganglion cells from degeneration after onset of diabetes. Retinal flatmounts harvested from diabetic mice injected with anti-sortilin antibody (middle column) compared to diabetic mice injected with PBS (right column) and non-diabetic mice (left column) after 8 weeks of diabetes (see FIG. 6 for experimental setup) and stained with DAPI. The number of retinal ganglion cells counted at 3 distinct locations in the periphery of each retina are shown in the graph. Mean values for each mouse are shown for non-diabetic control mice with black squares, diabetic mice injected with PBS with grey triangles and diabetic mice injected with anti-sortilin with white circles. *Statistically significant. DM: diabetes mellitus.

### DETAILED DESCRIPTION

Definitions

As used herein, the singular forms “a”, “an” and “the” include plural referents unless the context clearly states otherwise. Thus, for example, reference to “an antibody” includes a plurality of such antibodies, such as one or more antibodies, at least one antibody, or two or more antibodies.

“Inhibition” as used herein means that the presence of the antibody of the invention inhibits, in whole or in part, the binding of ligands to the receptor and/or the disablement of a signal the receptor would elicit upon ligand binding. This includes for example down-stream signaling having effect on cellular behavior and processes.

By “an antibody or an antigen-binding fragment thereof” we include substantially intact antibody molecules, as well as chimaeric antibodies, humanised antibodies, isolated human antibodies, single chain antibodies, bispecific antibodies, antibody heavy chains, antibody light chains, homodimers and heterodimers of antibody heavy and/or light chains, and antigen-binding fragments and derivatives of the same. Suitable antigen-binding fragments and derivatives include, but are not necessarily limited to, Fv fragments (e.g. single chain Fv and disulphide-bonded Fv), Fab-like fragments (e.g. Fab fragments, Fab′ fragments and F(ab)₂ fragments), single variable domains (e.g. V_{H} and V_{L} domains) and domain antibodies (dAbs, including single and dual formats [i.e. dAb-linker-dAb]). The potential advantages of using antibody fragments, rather than whole antibodies, are several-fold. The smaller size of the fragments may lead to improved pharmacological properties, such as better penetration of solid tissue. Moreover, antigen-binding fragments such as Fab, Fv, ScFv and dAb antibody fragments can be expressed in and secreted from E. coli, thus allowing the facile production of large amounts of the said fragments.

The phrase “an antibody or an antigen-binding fragment thereof” is also intended to encompass antibody mimics (for example, non-antibody scaffold structures that have a high degree of stability yet allow variability to be introduced at certain positions). Those skilled in the art of biochemistry will be familiar with many such molecules, as discussed in Gebauer & Skerra, 2009, Curr Opin Chem Biol 13(3): 245-255 (the disclosures of which are incorporated herein by reference). Exemplary antibody mimics include: affibodies (also called Trinectins; Nygren, 2008, FEBS J, 275, 2668-2676); CTLDs (also called Tetranectins; Innovations Pharmac. Technol. (2006), 27-30); adnectins (also called monobodies; Meth. Mol. Biol., 352 (2007), 95-109); anticalins (Drug Discovery Today (2005), 10, 23-33); DARPins (ankyrins; Nat. Biotechnol. (2004), 22, 575-582); avimers (Nat. Biotechnol. (2005), 23, 1556-1561); microbodies (FEBS J, (2007), 274, 86-95); peptide aptamers (Expert. Opin. Biol. Ther. (2005), 5, 783-797); Kunitz domains (J. Pharmacol. Exp. Ther. (2006) 318, 803-809); affilins (Trends. Biotechnol. (2005), 23, 514-522); affimers (Avacta Life Sciences, Wetherby, UK).

Persons skilled in the art will appreciate that the invention also encompasses modified versions of antibodies and antigen-binding fragments thereof, whether existing now or in the future, e.g. modified by the covalent attachment of polyethylene glycol or another suitable polymer.

Persons skilled in the art will further appreciate that well known methods for the production of polyclonal or monoclonal antibodies can be employed for the production of anti-sortilin antibodies.

Diabetes Mellitus (DM)

DM is a collective term for glucose metabolism disorders of different aetiology and symptoms with relative or absolute insulin deficiency and hyperglycemia as a common hallmark. It is the most common endocrine disease in the world with a continuously increasing trend.

Diabetic Retinopathy (DR)

DR is a disease of the fundus as a late consequence of diabetes mellitus. Damage to small blood vessels (microangiopathy) causes defects in the retina, which leads in its most severe form to blindness. Due to a lack of early symptoms, and thereby a delay of diagnosis and treatment, the development of prophylactic treatments is needed.

Retina

The retina is a multilayer tissue lining the eye between the choroid and the vitreous. The retina receives light impulses via the rods and cones serving as photoreceptors, converts them into minute electrical currents and passes them to the visual centers in the brain. The choroid is the vascular layer located external to the retina of the eye. The vitreous body is defined as the clear gel that fills the space between the lens and the retina of the eyeball of humans and other vertebrates.

The retina is built up of a multitude of cells forming a complex network, wherein different layers can be recognized. The different retinal layers encompass the nerve fiber layer (NFL), the ganglion cell layer (GCL), the inner plexiform layer (IPL), the inner nuclear layer (INL), the outer plexiform layer (OPL), the outer nuclear layer (ONL), the outer limiting membrane (OLM) and the inner and outer segments of the photoreceptors (PR I/O).

Müller cells are a special form of astroglia in the retina. In addition to the support function, they also take care of metabolic tasks.

Anti-Sortilin Antibody

The present invention relates to the inhibition of sortilin to protect retinal neurons from degeneration.

Therefore, in a main aspect the present invention relates to a composition comprising or consisting of an anti-sortilin antibody or an antigen binding fragment thereof, for use in the treatment or prevention of a disease or disorder of the retina, the choroid and/or the optic nerve.

In one embodiment the antibody or antigen binding fragment thereof is a polyclonal antibody or an antigen binding fragment thereof.

The usage of polyclonal antibodies might be useful since they bind to several binding sites, thereby affecting the binding of several ligands and potentially inhibiting several downstream pathways.

In another embodiment the antibody or antigen binding fragment thereof is a monoclonal antibody or an antigen binding fragment thereof.

The person skilled in the art will appreciate that any monoclonal antibody, available now or in the future, or newly generated, can be evaluated for its use in the treatment or prevention of a disease or disorder of the retina, the choroid and/or the optic nerve. Examples 2 and 3, using two different polyclonal antibodies (ab16640 and AF2934, respectively), show the implication of sortilin in the diabetic retina, and that treatment with anti-sortilin antibodies can limit retinal damage in a diabetic mouse intervention study.

In another embodiment commercially available monoclonal anti-sortilin antibodies are screened for their use in the treatment or prevention of a disease or disorder of the retina, the choroid and/or the optic nerve.

The usage of monoclonal antibodies might be useful since they bind to specific binding sites thereby inhibiting access of other molecules to this specific site.

It is understood by the person skilled in the art that antibodies can be obtained from different species.

In one embodiment the antibody or antigen binding fragment thereof is a human anti-sortilin antibody or an antigen binding fragment thereof.

In another embodiment the antibody or antigen binding fragment thereof is a goat anti-sortilin antibody or an antigen binding fragment thereof, or a rabbit anti-sortilin antibody or an antigen binding fragment thereof.

It is understood by the person skilled in the art that any antibody isotype might be used as an anti-sortilin antibody such as IgG, IgM, IgD, IgA or IgE.

In one embodiment the antibody or antigen binding fragment thereof is an IgG anti-sortilin antibody or an antigen binding fragment thereof.

Antibody Binding Sites

Anti-sortilin antibodies might bind against any sequence of sortilin and elicit an effect, for example an inhibitory effect. This might be due to, for example, and not excluding other possibilities, the inhibition of the binding of known ligands to their respective binding site, the inhibition of the binding of so far unknown ligands, the inhibition of the formation of complexes with co-receptors or alterations in the confirmation of the receptor.

In one embodiment the antibody or antigen binding fragment thereof binds to at least one amino acid of the amino acid sequence of SEQ ID NO. 1.

In another embodiment the antibody or antigen binding fragment thereof binds to at least one amino acid of the amino acid sequence of SEQ ID NO. 2.

Several ligands are known to bind to the extracellular domain of sortilin. The blocking of any of these binding sites is expected to have an inhibitory effect on the downstream signalling and thereby on cellular behaviour. Neurotensin binding sites and proNGF binding sites on sortilin have been identified.

In an embodiment the antibody or antigen binding fragment thereof binds to at least one amino acid of the amino acid sequence of SEQ ID NO. 3.

In another embodiment the antibody or antigen binding fragment thereof binds to at least one amino acid of the amino acid sequence of SEQ ID NO. 4.

In another embodiment the antibody or antigen binding fragment thereof binds to at least one amino acid of the amino acid sequence TGL, wherein said amino acid sequence is comprised in the Neurotensin binding site 3.

In another embodiment the antibody or antigen binding fragment thereof binds to at least one amino acid of the amino acid sequence of SEQ ID NO. 5.

In another embodiment the antibody or antigen binding fragment thereof binds to at least one amino acid of the amino acid sequence of SEQ ID NO. 6.

In another embodiment the antibody or antigen binding fragment thereof binds to at least one amino acid of the amino acid sequence of SEQ ID NO. 7.

In another embodiment the antibody or antigen binding fragment thereof binds to at least one amino acid of the amino acid sequence of SEQ ID NO. 8.

In another embodiment the antibody or antigen binding fragment thereof binds to at least one amino acid of the amino acid sequence of SEQ ID NO. 9.

In another embodiment the antibody or antigen binding fragment thereof binds to at least one amino acid of the amino acid sequence of SEQ ID NO. 10.

In another embodiment the antibody or antigen binding fragment thereof binds to at least one amino acid of the amino acid sequence of SEQ ID NO. 11.

In another embodiment the antibody or antigen binding fragment thereof binds to at least one amino acid of the amino acid sequence of SEQ ID NO. 12.

Antibody Variations

In another embodiment the antibody or antigen-binding fragment thereof is selected from the group consisting of Fab, F(ab′)₂, Fv fragments, Fab-like fragments, single variable domains, and nanobodies.

Further antibody variations can be found in the definition section.

In another embodiment the antibody or antigen binding fragment thereof is humanised.

In another embodiment the antibody or antigen binding fragment thereof is conjugated to a cell-penetrating peptide such as TAT-peptide.

The antibody or antigen binding fragment thereof can be modified or conjugates with molecules such as a TAT peptide. This will allow the antibody to penetrate cells which is advantageous for the usage of the composition in eye drops.

Treatment of Diseases of the Retina

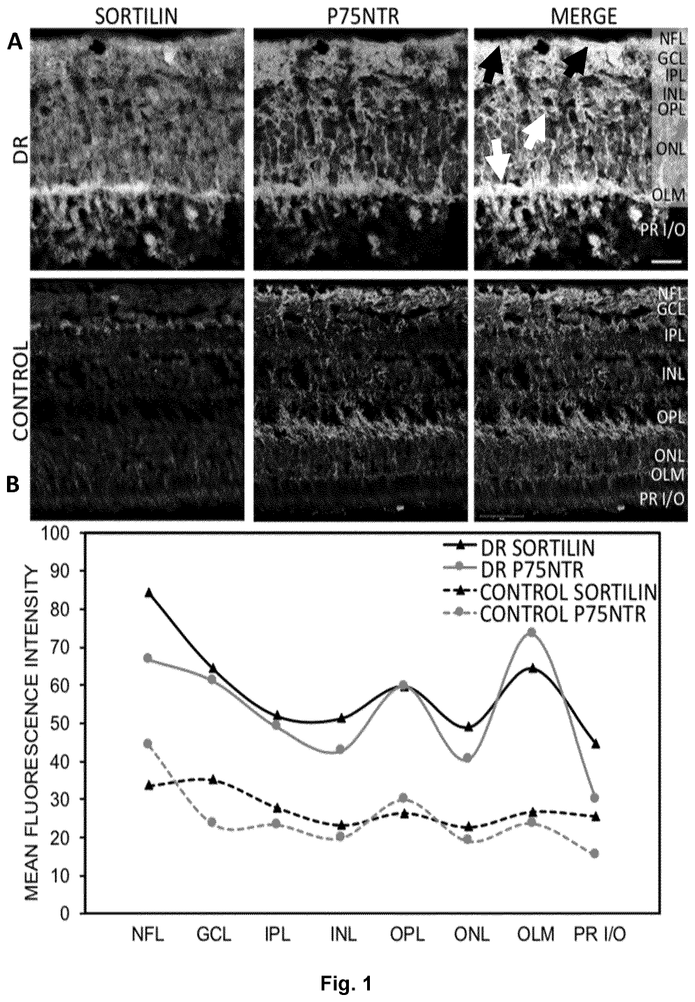

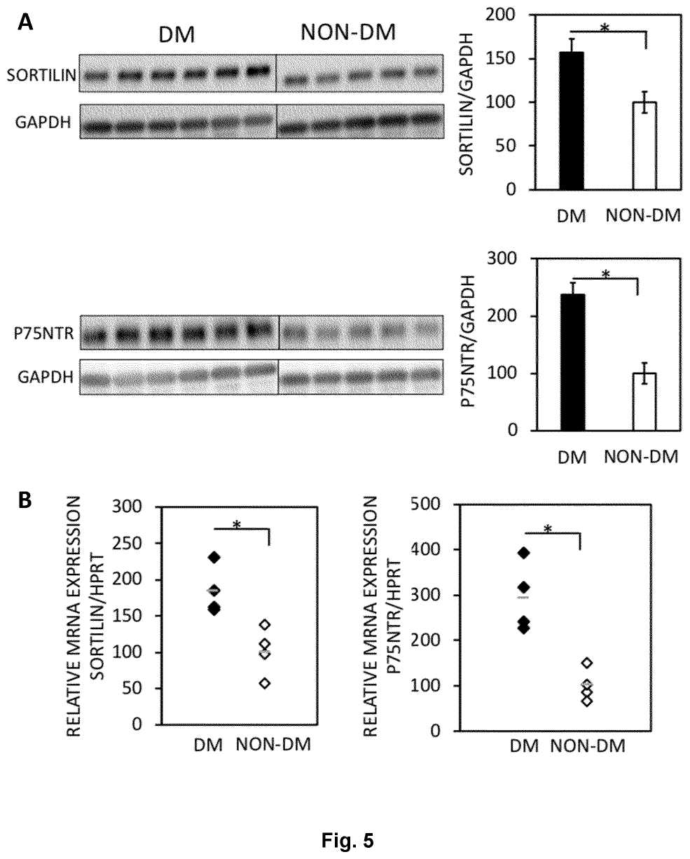

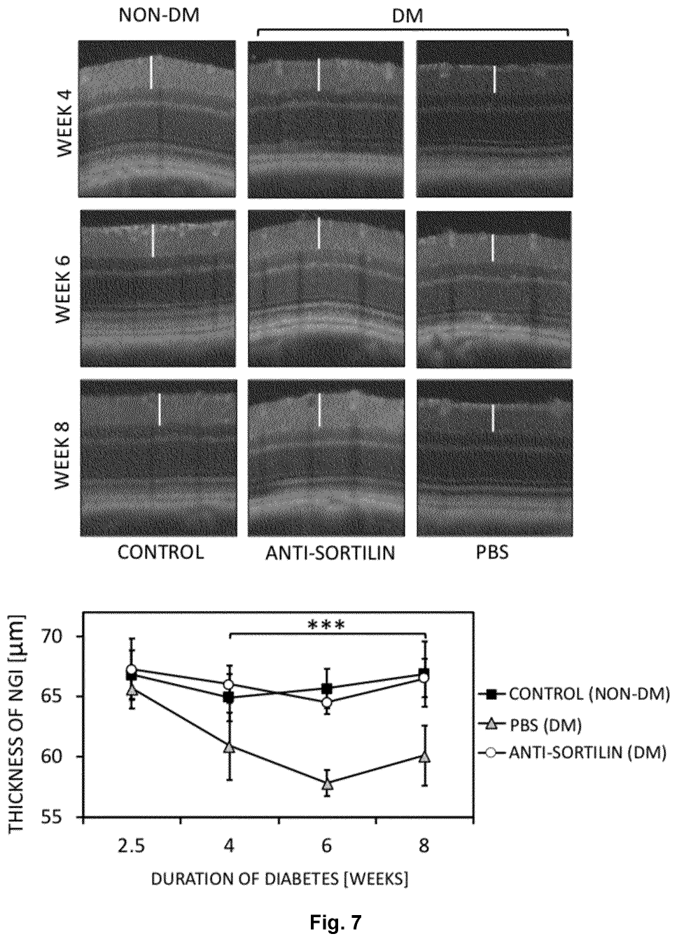

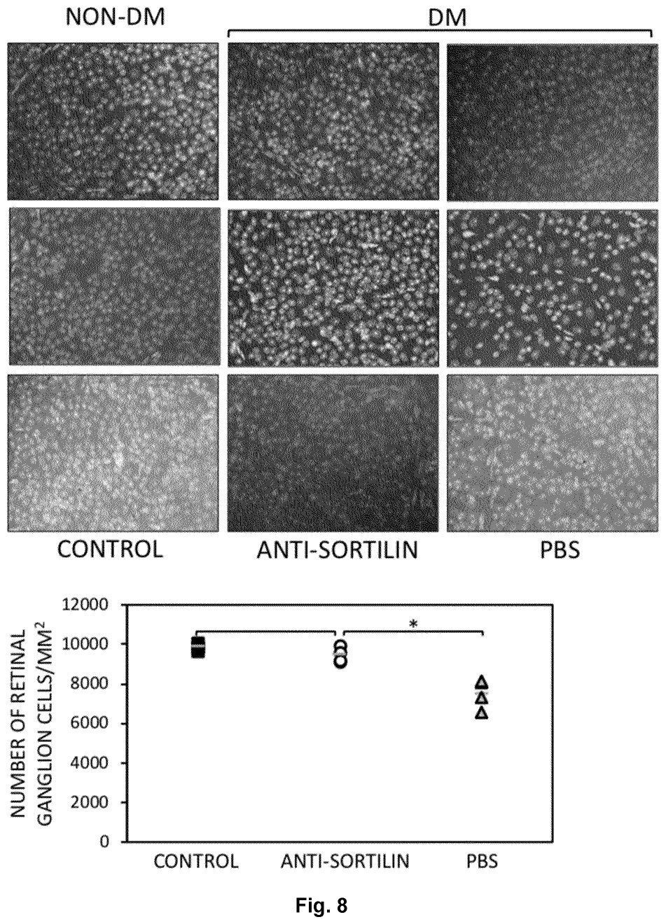

In the present invention it is shown that sortilin is overexpressed in DR patients with DM (FIG. 1) and in a diabetic murine retinas (FIG. 5). Administration of anti-sortilin protects the inner retina from degeneration after onset of diabetes (FIGS. 7 and 8).

Sortilin could play an essential role as a molecular switch, enabling proNGF-stimulated cells co-expressing p75^{NTR} to selectively undergo apoptosis or induce inflammation. Due to the proposed mechanism of sortilin as an important co-receptor for neuronal death signalling in several neurodegenerative diseases, it is understood that inhibition of sortilin can also protect from degeneration in eye diseases with similar aetiology. Diseases or disorders with such an alternative aetiology are, for example, diseases or disorders associated with light induced retinal damage or increased intraocular pressure (10P).

In an important embodiment the disease is a disease of the retina.

In a preferred embodiment the disease of the retina is diabetic retinopathy.

In a further embodiment the disease is selected from the group consisting of diabetic retinopathy, retinal vein occlusion, retinal artery occlusion, retinopathy of prematurity, Coats' disease, tapetoretinal degeneration, hereditary retinal dystrophy or any combination thereof.

In one embodiment the disease is hereditary retinal dystrophy.

The person skilled in the art will understand that the term hereditary retinal dystrophy can be understood synonymous to the term “inherited retinal dystrophy”. Both hereditary retinal dystrophy and inherited retinal dystrophy can also be used in plural as hereditary retinal dystrophies and inherited retinal dystrophies, comprising a diverse group of progressive blinding genetic diseases (Henderson 2020).

In another embodiment the disease is a disease of the choroid.

In another embodiment the disease of the choroid is selected from the group consisting of age-related macular degeneration, choroiditis or any combination thereof.

In another embodiment the disease is a disease of the optic nerve.

In another embodiment the disease of the optic nerve is selected from the group consisting of glaucoma, autosomal dominant optic neuropathy, Leber's hereditary optic neuropathy, optic neuritis, anterior ischemic optic neuropathy, toxic diseases of the optic nerve and nerve head or any combination thereof.

In another embodiment the disease or disorder is retinal degeneration or retinal tissue loss.

In another embodiment, the disease is a disease of the retina, a disease of the choroid or a disease of the optic nerve, or any combination thereof.

In another embodiment the composition is for use in the treatment of a mammal.

In another embodiment the composition is for use in the treatment of a human.

Administration of the Anti-Sortilin Antibody

It will be understood by the person skilled in the art that certain formulations are preferred for the delivery of a pharmaceutically active component to the eye, such as an anti-sortilin antibody. Local administration is beneficial since it ensures a sufficiently high concentration of the active component while at the same time avoiding systemic side effects.

It will be understood that the anti-sortilin antibody or an antigen binding fragment thereof can be administered in a pharmaceutically-acceptable diluent, carrier, adjuvant or excipient. This composition can be administered topically, for example in a gel, in a cream or in eye drops. Further, the antibody can be administered by a patch or by direct application, e.g. intravitreally, to the eye or by iontophoresis. The antibody may also be provided in sustained release compositions. The use of immediate or sustained release compositions depends on the nature of the condition being treated. If the condition consists of an acute or over-acute disorder, treatment with an immediate release form will be preferred over a prolonged release composition. Alternatively, for certain preventative or long-term treatments, a sustained release composition may be appropriate.

The anti-sortilin antibody or an antigen binding fragment thereof may also be delivered using an intraocular implant. Such implants may be biodegradable and/or biocompatible implants, or may be non-biodegradable implants. The implants may be permeable or impermeable to the active agent, and may be inserted into a chamber of the eye, such as the anterior or posterior chambers or may be implanted in the sclera, transchoroidal space, or an avascularized region exterior to the vitreous.

Antibody formulations which are given to the outside of the eye, for example in the form of eye drops or in the form of a gel, can be modified to improve the penetration into the eye. This can for example be achieved by conjugation with cell-penetrating peptides like the TAT-peptide and other modifications (Habault 2019).

When administered directly to the eye, for example by injection using a syringe, the dosage may be administered as a single dose or divided into multiple doses. The desired dosage could be administered at set intervals for a prolonged period, for example over several weeks, although longer periods of administration of several months or more may be needed.

Formulations suitable for intravitreal administration include aqueous sterile injection solutions which may contain anti-oxidants, buffers, bacteriostats and solutes. The formulations may be presented in unit-dose or multi-dose containers, for example sealed ampoules and vials, and may be stored in a freeze-dried (lyophilised) condition requiring only the addition of the sterile liquid carrier, for example water for injections, immediately prior to use.

In one embodiment the antibody or antigen binding fragment thereof is administered intravitreally.

This administration route encompasses the application of the antibody in a suitable form into or around the eye, for example by injection with a syringe.

In another embodiment the antibody or antigen binding fragment thereof is administered in the form of eye drops.

In another embodiment the antibody or antigen binding fragment thereof is administered topically on the eye.

In another embodiment the antibody or antigen binding fragment thereof is administered in the form of a gel.

Methods and Uses of an Anti-Sortilin Antibody

A further aspect of the present invention relates to the composition comprising or consisting of an anti-sortilin antibody or an antigen binding fragment thereof, for the manufacture of a medicament for the treatment or prevention of a disease of the retina, the choroid and/or the optic nerve.

This aspect and the further following aspects and embodiments relate to the composition comprising or consisting of an anti-sortilin antibody or an antigen binding fragment thereof as described above.

A further aspect of the present invention relates to a method of treatment of a disease of the retina, the choroid and/or the optic nerve, the method comprising administering to an individual in need thereof a therapeutically effective amount of an anti-sortilin antibody or an antigen binding fragment thereof.

A further aspect of the present invention relates to the composition comprising or consisting of an anti-sortilin antibody or an antigen binding fragment thereof, for use in the diagnosis of a disease of the retina, the choroid and/or the optic nerve.

The person skilled in the art will understand that the term antibody or antigen binding fragment thereof in the further aspects of the present invention includes the antibody variations as elaborated above.

In an embodiment the sortilin antibody or antigen binding fragment thereof is used in an ELISA for the detection of soluble sortilin.

Soluble sortilin could be detected in for example tear fluid and in tissue samples such as aqueous and vitreous humour samples.

In another embodiment the sortilin antibody or antigen binding fragment thereof is used for detection of sortilin expression tissue samples by antibody based staining such as immunohistochemistry and/or immunofluorescence.

A further aspect of the present invention relates to a method for detecting a disease of the retina, the choroid and/or the optic nerve by using an anti-sortilin antibody or an antigen binding fragment thereof, said method comprising analysing in a sample obtained from a mammalian retina, choroid or optic nerve, the presence or absence of an antigen comprising a sortilin polypeptide, wherein the presence of the antigen is indicative of a disease of the retina, the choroid and/or the optic nerve.

A further aspect of the present invention relates to a method for preventing retinal degeneration or retinal tissue loss, said method comprising administering a composition comprising an anti-sortilin antibody or an antigen-binding fragment thereof.

In one embodiment the effect of preventing retinal neurodegeneration is analysed by the measurement of
a) retinal layer thickness and/or
b) the number of retinal neural cells.

A further aspect of the present invention relates to a method for preventing apoptosis of cells in the retina, the choroid and/or the optic nerve, said method comprising administering a composition comprising an anti-sortilin antibody or an antigen-binding fragment thereof.

### EXAMPLES

### Example 1

### Sortilin Expression and Localization in the Human Diabetic Retina

Aim:

This example shows the expression and localization of sortilin in non-diabetic and diabetic human retinas

Materials and Methods:

Immunofluorescence staining of paraffin sections: paraffin sections were obtained from six patients with a verified history of diabetic retinopathy and from eyes from five normal controls. Paraffin was removed by placing the sections in xylene overnight (ON) and rehydration in a graded ethanol series. Slides were washed in water before antigen retrieval was performed in TE buffer pH 9 (HIER). Sections were blocked in 2% BSA before incubation with primary antibody in 1% BSA ON at 4° C. Antibodies used were anti-sortilin (ab16640; Abcam) 1:200; anti-GFAP (C-19) (sc-6170; Santa cruz) 1:250; anti-NGFR (BD-557194; BD biosciences) and anti-GS (MAB-302; Millipore) 1:1000. Secondary antibodies (Alexa fluor 488 goat anti-mouse, Alexa fluor 568 donkey anti-rabbit, and Alexa fluor 488 donkey anti-goat) were diluted 1:400 and incubated for 1 hour at RT. Nuclei were stained with 4′,6-diamidino-2-phenylindole (DAPI).

Microscopy: Fluorescence staining and co-localization was examined using a Zeiss LSM 710 confocal laser scanning microscope (CLSM) (Zeiss, Jena, Germany) fitted with a Plan-Apochromat 20×63×1.4 objective. Excitation and emission wavelengths for Alexa Fluor 488 were λₑₓ=488 nm and λₑₘ 525 nm, and correspondingly for TRITC: λₑₓ=532 nm and λₑₘ=576 nm.

Quantification of immunofluorescence intensity: Vertical densitometric profile plots representing the mean fluorescent signal intensity across the retinas in each group were generated using the Plot Profile function of ImageJ for both sortilin and p75^{NTR} utilizing the DAPI nuclear stain to determine the location in the retina. Three linear plot profiles were analysed for each retinal image and the mean fluorescent signal intensity was calculated. In each of the retinal images, 8 different retinal layers were identified: optic fiber layer (OFL), ganglion cell layer (GCL), inner plexiform layer (IPL), inner nuclear layer (INL), outer plexiform layer (OPL), outer nuclear layer (ONL), outer limiting membrane (OLM) and inner and outer segments of the photoreceptors (PR I/O). For each layer, the mean fluorescent signal intensity was calculated. For each group (diabetes and controls), the mean fluorescent signal intensity for each of the 8 layers has been depicted.

Results:

Elevated sortilin and p75^{NTR} levels in patients with diabetic retinopathy vs. healthy controls: Sortilin and p75^{NTR} were detectable in all human retinas examined (FIG. 1A). Densitometric profile analysis of the fluorescent signals reveals considerably higher levels of sortilin and p75^{NTR} in the retina of diabetic patients compared to non-diabetic controls (FIG. 1B). In the diabetic retinas, the fluorescent signal for sortilin is strongest in the OFL in contrast to the non-diabetic control patients, where the highest signal was found in GCL. The highest level of p75^{NTR} was observed in the OLM in diabetic retinas in contrast to the non-diabetic control retinas, where the highest levels were located in the OFL. Notably, the sortilin and p75^{NTR} receptors covary in the retina.

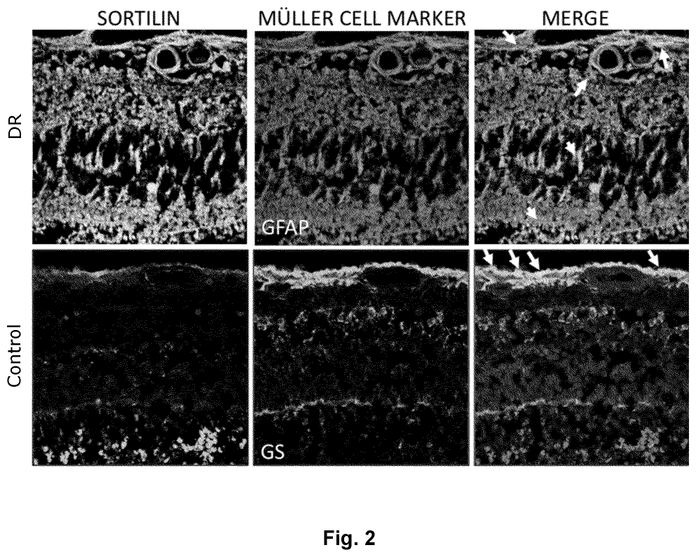

Sortilin co-localizes with p75^{N} and GFAP in patients with diabetic retinopathy: Assessment of the localization pattern of sortilin in the retina revealed the highest levels of sortilin in the inner retina, but spanning the entire retina in the diabetic patients with DR. Co-immunostaining of sortilin and p75^{NTR} shows co-localization of the two receptors in retinal tissue from DR-patients, particularly in the inner retina (FIG. 1A, arrows). In contrast, no co-localization was found in either part of the retina in the non-diabetic samples (FIG. 1A). Co-immunostaining for GFAP/GS and sortilin revealed co-localization, which is most evident in the DR-patients due to the high expression of GFAP in these retinas (FIG. 2 arrows).

Conclusions:

Sortilin levels are considerably increased and co-localize with p75^{NTR} in the retina from patients with diabetic retinopathy. Sortilin co-localizes with Muller cell specific markers in the human retina (GFAP and GS).

### Example 2

### Sortilin Expression and Localization in the Retina of Diabetic Mice

Aim:

This example shows the expression and localization of sortilin in retinas from non-diabetic and diabetic mice.

Materials and Methods:

Diabetes induction: Diabetes was induced in 8 weeks old C57BI6/J male mice by five intraperitoneal streptozotocin (STZ) injections on five consecutive days using doses of 55 mg/kg body weight. Blood glucose was measured in tail-capillary blood by Contour. Body weight and blood glucose were measured weekly throughout the study. Animals were considered as diabetic when blood glucose was above 15 mmol/L.

Paraffin embedding and sectioning: After 20 weeks of diabetes, eyes were enucleated and fixed in 4% paraformaldehyde. Eyes were dehydrated and embedded in paraffin. Sagittal sections of 3 μm were prepared.

Immunofluorescence staining of paraffin sections: Performed as described in Example 1.

Microscopy: Performed as described in Example 1.

Western blotting: Mice were sacrificed after 20 weeks of diabetes by cervical dislocation. Eyes were extracted and retinas processed for Western blotting as described previously (Askou 2019). Western blotting was performed as described previously (Askou 2017). Membranes were incubated at 4° C. ON with rabbit anti-sortilin antibody (ab16640; Abcam) or goat anti-p75^{NTR} antibody (AF1157, RD systems) in a concentration of 1:5000 or 1:1000, respectively. As a loading control, membranes were incubated with mouse anti-GAPDH (G8795, Sigma Aldrich) in a concentration of 1:10,000. Visualisation and quantification were done as described previously (Askou 2019). Statistical differences between two groups were evaluated using Student's t test. A p value of <0.05 was considered statistically significant.

RT-PCR: Mice were sacrificed after 20 weeks of diabetes by cervical dislocation. Eyes were enucleated and dropped in ice-cold HBSS buffer. Periocular tissue was removed and the anterior segment and lens were discarded. The neuroretina was dropped in ice-cold lysis buffer (buffer RLT+40 mM DTT). The tissue lysate was loaded onto a QIAshredder for homogenisation and subsequently, RNA was purified using the RNeasy plus kit, according to protocol (QIAGEN). The iScript cDNA synthesis kit (Bio-Rad) was used for first-strand cDNA synthesis, with a total input RNA of 50 ng, according to the protocol. PCR reactions were performed in triplicates on 1:5 diluted cDNA using Taqman™ Gene expression assays and analysed on a LightCyclerW480 (Roche Diagnostics) according to Taqman™ protocols. Relative gene expression of sortilin (Mm00490905; ThermoFisher Scientific) and p75^{NTR} (Mm00446296; ThermoFisher Scientific) was calculated using the standard curve method and related relative to an endogenous control, the housekeeping gene hypoxanthine-guanine phosphoribosyl transferase (HPRT; Mm00446968; ThermoFisher Scientific). Statistical differences between two groups were evaluated using Student's t test. A p value of <0.05 was considered statistically significant.

Results:

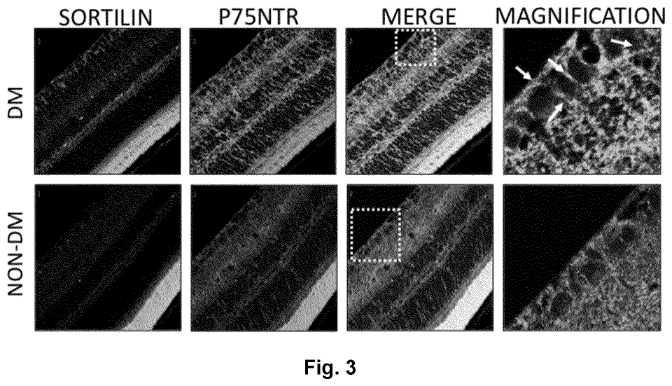

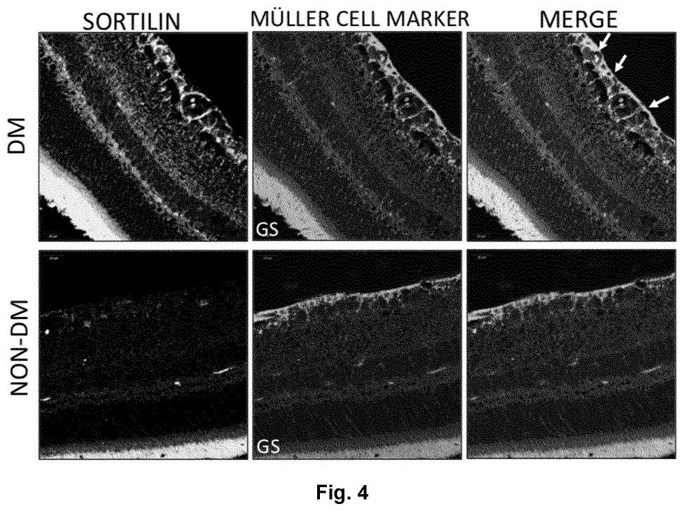

Sortilin co-localizes with p75^{NTR} and GS in retinas of diabetic mice: Sortilin and p75^{NTR} were detectable in all murine retinas examined (FIGS. 3 and 4). Assessment of the localization pattern of sortilin in the retina of diabetic mice (FIGS. 3 and 4), reveals the highest levels of sortilin in the inner retina, but spans the entire retina, resembling the pattern in diabetic patients. Co-immunostaining of sortilin and p75^{NTR} shows a high degree of co-localization of the two receptors in retinal tissue from diabetic mice, particularly in the inner retina (FIG. 3, arrows). Co-immunostaining for GS and sortilin revealed co-localization, which is most evident in the diabetic retinas due to the high expression of sortilin in these retinas (FIG. 4).

Elevated sortilin and p75^{NTR} levels in retina of diabetic mice: Quantification of total protein levels of sortilin and p75^{NTR} by Western blotting revealed increased amounts in the diabetic murine retinas compared to the non-diabetic ones, with an increase of 58% and 138%, respectively (FIG. 5A). This increase was also found on the mRNA level, where sortilin mRNA expression was increased by 84% and p75^{NTR} by 194% in the diabetic retinas (FIG. 5B).

Conclusion:

The expression and localization pattern of sortilin and p75^{NTR} found in diabetic murine retinas corresponds to the findings in human diabetic retinas. Sortilin and p75^{NTR} expression are increased in the diabetic retinas compared to non-diabetic controls and this increase is reflected on protein levels as well. The STZ-induced diabetic mouse is a good model for investigation of sortilin inhibition in diabetic retinas.

### Example 3

### Intervention Study in the STZ-Induced Diabetic Mouse

Aim:

This example aims at elucidating the effects of administering anti-sortilin antibodies in a disease model of retinal neurodegeneration.

Materials and Methods:

Diabetes induction: Diabetes was induced in 20 eight weeks old C57BI6/J male mice as described in Example 2. 10 non-diabetic control mice were injected intraperitoneally (IP) with buffer only.

Intervention study: At T_{2.5} (2.5 weeks after onset of DM), mice were anesthetized with a combination of ketamine and medetomidin hydrochloride (Ketador 60-100 mg/kg and Cepetor 0.5-1 mg/kg), pupils dilated with a drop of 1% tropicamide and OCT (optical coherence tomography; a non-invasive imaging technique routinely used by ophthalmologists in order to validate the health of the retina. OCT uses low-coherence light to capture cross-section pictures of high resolution) images were obtained from all 30 murine retinas by the Micron IV image-guided OCT 2 (Phoenix Research Laboratories). Subsequently, the 20 diabetic mice were injected intravitreally (IVT) with 2 μL of 1 mg/mL goat anti-sortilin polyclonal antibody (pAb) (AF2934; RD Systems) in the left eye and with 2 μL PBS IVT in the right eye. During anesthesia, eyes were lubricated with a carbomer eye gel (Viscotears 2 mg/g). Immediately after the investigations, mice were brought out of anesthesia with Antisedan 0.5-1 mg/kg, and placed on a warming plate until they moved spontaneously. Image-guided OCT images were obtained at T₄, T₆ and T₈ (4, 6 and 8 weeks after onset of DM, respectively) in the central retina at an equal distance from the optical disc (OD).

Determination of thickness of NGI (Nerve fiber layer, Ganglion cell layer and Inner plexiform layer): OCT images were loaded into the InSight software (Phoenix Research Laboratories) and average thickness of the NGI was determined by segmenting retinal layers manually.

Preparation of retinal flatmounts: At T₈, mice were sacrificed and eyes were enucleated and fixed in 4% paraformaldehyde for 2 H at room temperature. Periocular tissue was removed and the anterior segment and lens were discarded. The neuroretina was carefully peeled of the RPE/choroid and immersed in PBS-buffer. Retinas were permeabilized and blocked in 1% BSA and 1% Triton X-100 in PBS ON at 4° C. on a shaker. Retinas were washed before and after addition of DAPI. Finally, flat-mounts were gently transferred to Super-FrostPlus glass slides, mounted with the RGC (retinal ganglion cell) layer facing upwards using ProLong Gold antifade reagent (Life Technologies).

Image acquisition: Images for assessment of RGC (retinal ganglion cell) densities were acquired in the periphery (1 mm from the OD) by fluorescence microscopy (Leitz DM RB; Leica Microsystems). Images were captured with a Leica DFC 360 FX camera and associated software (Leica Application Suite version 3, Leica Microsystems). The RGC layer was placed in the focal plane and the number of RGC was counted manually on three images from each retina and a mean was calculated (n=4 retinas in each group). Statistical differences between two groups were evaluated using Student's t test. A p value of <0.05 was considered statistically significant.

Results:

Protective Effect of Anti-Sortilin Treatment:

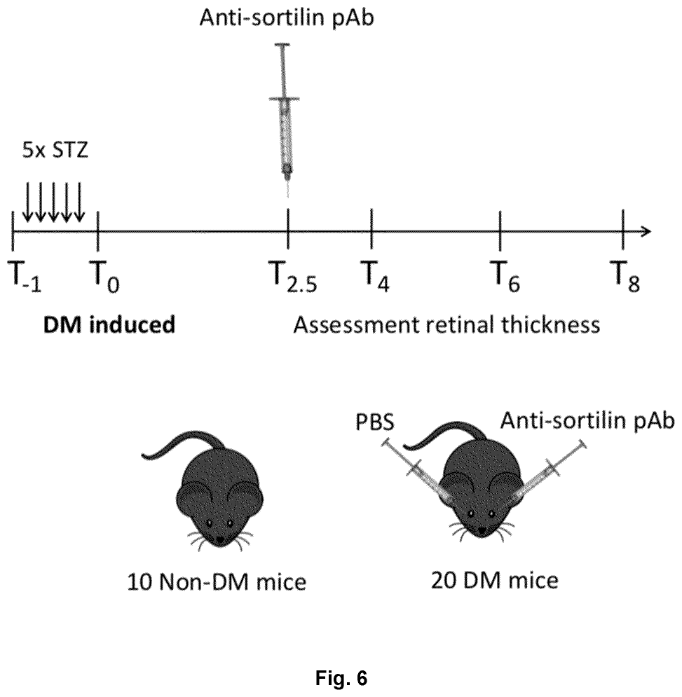

The protective effect of anti-sortilin against retinal neurodegeneration in the inner retina was investigated by injecting 2 μg anti-sortilin antibody IVT at 2.5 weeks after induction of diabetes in C57B16/J mice. Anti-sortilin antibodies were injected IVT in the left eye of 20 mice (FIG. 6). As controls, DM mice were injected IVT with PBS in the right eye and age-matched non-DM mice were included. The retina was imaged in a longitudinal study by OCT performed at T_{2.5}, T₄, T₆, and T₈. The B-scans were obtained as a circle scan at a distance of 0.25 mm from the center of the optic disc. In each B-scan, the Nerve fiber layer, the Ganglion cell layer and the Inner plexiform layer (herein termed NGI) was measured, as an estimate of the density of the RGC cell bodies and fibers (FIG. 7). Using the Phoenix Insight semi-automated segmentation software, the NGI thickness is calculated as a mean value of hundreds of measurements along the circular B-scan. The thickness of the NGI was significantly reduced by 8% in the eye of the diabetic mice injected IVT with PBS compared to the eye injected with anti-sortilin pAb 4 weeks after onset of diabetes (p=0.0003; n=8 and n=9 eyes IVT anti-sortilin and PBS, respectively). At T₆, this reduction reached 10% (p=0.00006; n=5 and n=4 eyes IVT anti-sortilin and PBS, respectively), after which no further reduction was seen. The therapeutic efficacy achieved by IVT administration of a single injection of anti-sortilin pAb was maintained for up to week 8 of diabetes (e.g. for at least 5.5 weeks). At T₈, the NGI of diabetic anti-sortilin IVT mice measured 66.55 μm±1.59 μm compared to diabetic non-treated eyes 60.11 μm±2.49 μm (p=0.0000005; n=12 and n=11 eyes IVT anti-sortilin and PBS, respectively) and resembling the non-diabetic control mice with a thickness of 66.88 μm±2.71 μm. We quantified the number of RGCs in flatmounted whole retinas from 4 eyes in each group. Quantification was done at a specified distance from the OD to account for normal thinning of the RGC density toward the periphery. After 8 weeks of diabetes, there was a significant reduction in number of RGC of 24% (p=0.0008) in the PBS injected diabetic eyes compared to non-DM control eyes (FIG. 8). To investigate whether RGC loss in diabetic retinas is sortilin-dependent, mice were injected with anti-sortilin pAb at T_{2.5} as described above. Treatment with anti-sortilin pAb preserved the RGCs counted at T₈ of diabetes, and the 4% reduction in the number of RGC was insignificant (p=0.1; DM anti-sortilin vs. non-DM control).

Conclusion:

RGC loss in the diabetic retina is sortilin dependent and treatment of diabetic eyes with a single IVT injection of 2 μg anti-sortilin pAb at 2.5 weeks after onset of diabetes protected the NGI from thinning, by reducing RGC death, and preserved retinal structures.

### Example 4

### Sortilin Expression and Localization in Glaucoma

This example shows the expression and localization of sortilin in normal and glaucomatous retinas

Materials and Methods:

Sortilin expression and localization in the human glaucomatous retina is determined by immunofluorescence staining of paraffin sections obtained from patients with a verified history of glaucoma and from eyes from normal controls. Further, immunofluorescence intensity is quantified by densitometric profile plots across the retinas in each group. Co-localization of sortilin and p75NTR is assessed by co-staining of paraffin sections. The methodology as presented in Example 1 is applied.

Results:

Sortilin levels are expected to be increased and to co-localize with p75NTR in the inner retina from patients with glaucoma, whereas no co-localization of sortilin and p75NTR is expected to be found in the non-glaucomatous, age-matched samples. Furthermore, sortilin is expected to co-localize with Muller cell specific markers (GFAP and GS).

Conclusion:

Sortilin levels are expected to be considerably increased and to co-localize with p75NTR in the retina from patients with glaucoma, compared to eyes from normal controls. Further, sortilin is expected to co-localizes with Muller cell specific markers in the human retina (GFAP and GS). This expected importance and involvement of sortilin in glaucoma strengthens the usefulness of anti-sortilin antibodies or antigen binding fragments thereof, for use in the treatment, prevention, diagnosis and/or detection of glaucoma.

### Example 5

### Intervention Study for the Prevention/Treatment of Glaucoma

Aim:

This example aims at elucidating the effects of administering anti-sortilin antibodies in a disease model of glaucoma.

Materials and Methods:

Models for neuropathic diseases of the retina such as glaucoma that involve the death of retinal ganglion cells (RGC) include the widely used mouse model, where the retina is chronically stressed by increased intraocular pressure (IOP). Proof-of-concept studies are performed in sortilin knockout mice, where chronical retinal neurodegeneration is induced by episcleral vein cauterization surgery (Ruiz-Ederra 2006). In a time-kinetic study, retinal images are acquired by OCT and the NGI thickness is determined as described in Example 3. Electroretinogram (ERG) responses are also obtained, and the oscillatory potentials obtained from sortilin knockout mice with 10P are compared to WT control mice with IOP.

Mice are euthanized at day 42, where the number of surviving RGC's is quantified as described in Example 3.

In another experiment, experimental eyes from WT mice are injected intravitreally with anti-sortilin antibodies, whereas the contralateral eye is injected with control antibodies. Injections are performed at days 14 and 21 after induction, and the endpoint is 42 days. Retinal images are acquired by OCT, ERG analysis performed, and the number of surviving RGC's is quantified as described above and in Example 3.

Results:

Sortilin knockout, and sortilin inhibition by antibody injection, is expected to afford protection and survival of RGC in the setting of glaucoma. Evidence of this is expected to be: increased thickness in OCT images in sortilin knockout retinas compared to control retinas; increased number of RGC in retinal flatmounts from sortilin knockout retinas compared to control retinas; or ERG responses presenting with a higher oscillatory potentials contribution in sortilin knockout retinas compared to control retinas.

Conclusion:

Sortilin knockout, and sortilin inhibition by antibody injection, protects retinal ganglion cells from degeneration in a mouse model of chronic IOP. These findings support the usefulness of anti-sortilin antibodies or antibody fragments thereof for use in the treatment, prevention, diagnosis and/or detection of glaucoma.

### Example 6

### A Cellular Assay for the Identification of Anti-Sortilin Antibodies Capable of Inhibiting Binding of the Prongf Prodomain to Sortilin

Aim:

This example shows how to screen for inhibitory anti-sortilin antibodies, for example monoclonal antibodies, which are able to inhibit binding of ligands to sortilin.

Materials and Methods:

Reagents

Anti-sortilin antibodies, preferably monoclonal antibodies, are screened for their usefulness in the treatment, prevention, diagnosis and/or detection of a disease or disorder of the retina, the choroid and/or the optic nerve, for example diabetic retinopathy or glaucoma. This usefulness is, for example, assessed by the antibody's capability of inhibiting binding of ligands to sortilin. Novel anti-sortilin monoclonal antibodies are generated by methods known in the field and screened. Further, available anti-sortilin antibodies (for example, available in collaborations or produced in-house or commercially available), are screened. Examples of anti-sortilin monoclonal antibodies are the antibodies available from known antibody providers, for example Merck, Abcam, R&D Systems, ThermoFisher Scientific, Proteintech, LSBio, Cell Signaling Technology, Novus Biologicals, BD Transduction Laboratories or Sigma-Aldrich.

The expression construct for human proNGF is generated by cloning of a synthetic gene (residues 19-241 of P01138+6 C-terminal His residues, Geneart) in pcDNA 3.1 expression vector. Cultured media (1000 ml) from transient expression in CHO-S cells (ThermoFisher Scientific) is applied to a 5 ml HisTrap column and washed with 20 mM Sodium Phosphate pH 7.4, 1 M NaCl (A buffer). Elution of bound protein is done in a linear gradient to 0.25 M Imidazole in A-buffer over 20 column volumes and a flow of 5 ml/min. Fractions are analysed by SDS-PAGE and pooled based on proNGF content. Finally, the pool is dialysed against 1× PBS (Invitrogen) at 4° C. Samples are stored at −20° C. in aliquots. Recombinant human proNGF expressed and purified from E. coli is purchased from Alamone labs.

GSTpro is engineered as a fusion of Glutathione S-transferase (GST) merged at the C-terminal of GST to the pro part (19-121) of human proNGF. The construct is cloned into pGEX expression plasmid and used for expression in E. coli using the Overnight ExpressT Autoinduction System 1 (Novagen). The cells are harvested, lysed and from the supernatant the GSTpro is purified, using standard Glutathione-Sepharose affinity chromatography. Neurotensin and Neurotensin derived peptides are synthesized by GenScript Biotech.

Cell Culture for Sortilin Cell-Based Assay

HEK 293 cells are grown in DMEM with 10% fetal bovine serum. They are transfected with plasmids either encoding wild type sortilin, or sortilin with a mutation that renders it endocytosis deficient, or an empty control vector, according to manufacturer's instructions using 20 μg lipofectamine (Thermo Fischer Scientific) with 8 μg DNA on 4.5×10⁶ million cells per 6 cm, poly-lysine coated dish. The cells are initially plated into 24-well dishes after transfection. That intermediate step renders more uniform cell numbers in the 96-well dishes that are used to run the actual assay. 24 h later, cells are split into black opaque-walled, clear-bottom 96 well dishes at 42000 cells in 80 μl medium/well. 23 h after plating into 96 well dishes, cells are treated with different concentration of anti-sortilin antibodies to be tested for blocking sortilin-NGF pro-domain interaction, or blocking compounds, or control compounds, or neurotensin (positive control), or a scrambled neurotensin peptide (negative control), or a 4 mer or 3 mer peptide derived from the C-terminal part of neurotensin (positive control), or a reverse 3 mer C-terminal peptide of Neurotensin (negative control). 1 h after that treatment, the medium is replaced with 80 μl medium containing the same antibody included in the preincubation medium, plus recombinant GSTpro or proNGF (either purified in-house from recombinant HEK cells or derived from an E. coli expression system at either 0 nM (negative control), or 50 nM, or, in a few instances at 5 or 10 nM. Cells are washed twice with prewarmed PBS and fixed in 4% PFA for 20 min at approximately 20 or 37° C.

Immunocytochemistry

The fixed cells are washed with PBS for 15 min, followed by two 15 min washes with PBS with 0.1% Triton X-100. The cells are then treated with PBS with 10% FBS for 10 min and subsequently incubated with primary antibodies at 4° C. overnight as follows: To test expression of sortilin, control wells are stained with an anti-sortilin antibody at a 1:500 concentration in 10% FBS/PBS (Mouse IgG1 Anti sortilin, BD Transduction Laboratories™ number 612101). As some of the sortilin-pro domain blocking antibodies to be tested are mouse-derived, the use of secondary anti-mouse antibodies for immunohistochemical staining needs to be avoided if possible. Thus, in immunohistochemical staining, goat-derived anti-sortilin antibodies (1:800 affinity-purified polyclonal antibody BAF2934; R&D Systems) are used to test the blocking of sortilin-pro interaction by mouse antibodies. Wells to be evaluated for blocking of the sortilin-GSTpro interaction by antibodies are only stained with an antibody against the pro domain of proNGF in 10% FBS ata dilution of 1:1500 (Millipore (N-term) clone EP1318Y, Rabbit Monoclonal Antibody Catalog Number: #04-1142). To stain against GST, a rabbit anti-GST antibody is applied at 1:600 (abcam ab9085).

The following day, wells are washed 3×15 min with PBS/0.1% Triton X-100. The secondary antibodies are centrifuged at 13000 g for 2 min before dilution. All antibodies are diluted in PBS/10% FBS with 0.5 μg/ml Hoechst dye and filtered through a Millipore express MC 0, 22 μm syringe-attached Filter Unit. Cells that have will have been incubated with a mouse-derived anti-sortilin antibody are incubated with an Alexa 594 donkey anti-mouse antibody at a 1:3000 dilution. Wells that will previously have been incubated with an anti-proNGF antibody are subsequently incubated with an Alexa 488 donkey anti rabbit (A110034) antibody at a 1:400 dilution. To detect GSTpro, an Alexa488 goat anti-rabbit (ThermoFisher A11034) antibody is applied at a 1:300 dilution. Both secondary antibodies are applied for 1 h in the dark. Cells are then washed 1×15 min with PBS+0.1% Triton X-100, and 2×15 min with PBS. Cellular fluorescence is quantified using an array scanner and the “Neuronal Profiler” Bioapplication (Thermo Fisher Scientific).

Sortilin-GSTpro interaction is also assessed using HEK cell derived cell lines stably expressing sortilin. To this end, normal HEK cells (negative control) or cells transfected with full-length or truncated sortilin cells are directly plated into 96 well dishes and treated with antibodies or compounds 23 h later as described above.

To further examine whether GSTpro is internalized, rather than only binding to the cell exterior, extracellularly bound ligand is removed by washing cells in PBS acidified to pH 2.0 with HCl supplemented with 0.03 M sucrose and 10% FCS immediately before fixation.

Evaluation of Sortilin-Mediated GST-Pro Uptake Using Automated High Content Screening

To assess GST-pro binding and uptake to the “sortilin-positive” cell population and “sortilin-negative” cell population with no red staining in their vicinity, images from 96 well dishes are automatically recorded with a Cellomics ArrayScan VTI HCS Reader (Thermo Scientific) using a 10× microscope objective and the build-in standard autofocus method. Fifteen 1024×1024 images in 4 channels (Channel 1 and 2: Filter XF5-Hoechst, to detect nuclei and all cells; Channel 3: XF53-Texas Red to detect, e.g., sortilin-positive cells; Channel 4: Filter XF93-FITC, to detect cells positive, e.g., for the proNGF pro domain) are recorded per 96 well. The images are analyzed with the Cellomics assay algorithm NeuronalProfiling.V3.5. The inhibitory potential of an antibody is quantified as the percentage of GST-pro staining signal in the absence or presence of the antibody to be tested.

Results:

Inhibitory anti-sortilin antibodies, which are able to inhibit binding of ligands to sortilin, are identified.

Conclusion:

The identified inhibitory anti-sortilin antibodies can be used in the treatment, prevention, detection and/or diagnosis of a disease or disorder of the retina, the choroid and/or the optic nerve.

### Example 7

### Identification of Anti-Sortilin Antibodies Capable of Inhibiting proNGF Induced Apoptosis In Vitro and In Vivo.

Aim:

This example shows how to screen for inhibitory anti-sortilin antibodies, for example monoclonal antibodies, which are able to inhibit apoptosis.

Materials and Methods:

Reagents

Anti-sortilin antibodies, preferably monoclonal antibodies, are screened for their usefulness in the treatment, prevention, diagnosis and/or detection of a disease or disorder of the retina, the choroid and/or the optic nerve, for example diabetic retinopathy or glaucoma. This usefulness is, for example, assessed by the antibody's capability of inhibiting apoptosis associated with disease processes, such as the pro-apoptotic action of proNGF. Novel anti-sortilin monoclonal antibodies are generated and screened. Further, available anti-sortilin antibodies (for example, produced in-house or commercially available), are screened. Examples of anti-sortilin monoclonal antibodies are the antibodies available from known antibody providers, for example Merck, Abcam, R&D Systems, ThermoFisher Scientific, Proteintech, LSBio, Cell Signaling Technology, Novus Biologicals, BD Transduction Laboratories or Sigma-Aldrich.

661W cells that maintain photoreceptor phenotypes are obtained from transgenic mouse retinas expressing SV40 T antigen. Adult 60 days old female BALB/c albino mice are obtained from Envigo.

Induction of Light Damage

Albino mice are dark-adapted for 24 h before exposure for 7 h to cool white light (Master PL Electronic 23 W, 230-240 V Cool Daylight, Royal Philips Electronics, Amsterdam, Holland) at a luminescence level of 10,000 lux. The mice are then kept in complete darkness for 6, 12, or 24 h.

For light damage in 661W cells, cells are grown to 80% confluency in growth medium and then transferred to serum free medium, where they are cultured for 18 h before exposure for 3 h to cool white light (Master PL Electronic 23 W, 230-240 V Cool Daylight) at a luminescence of 15,000 lux. Cultures are then returned to the incubator for a further 2 h. Cells maintained under similar conditions but not exposed to illumination served as controls.

The 661W assay can be used as general screening assay for anti-apoptotic antibodies, regardless the disease and cell type. The advantage of this assay is that light-induced damage will induce expression of both sortlin, p75NTR and proNGF (Santos 2012).

Histology and Immunohistochemistry

Entire enucleated eyes from control and light-exposed animals (three mice for each experimental point) are fixed in periodate lysine paraformaldehyde for 6 h at 4° C. The fixed material is cryoprotected in PBS containing 30% sucrose, soaked in OCT compound (Sakura Finitek Europe, Zoeterwoude, The Nether-lands), and frozen in liquid nitrogen. Blocks are stored at 24° C. until use. Transverse sections (20 mm) are obtained in a cryostat (Leica, Wetzlar, Germany) and collected on SuperFrost slides (Menzel-Glasser, Braunschweig, Germany). Cryosections are permeabilized and blocked for 30 min at room temperature in PBS containing 0.5% Triton X-100 (Sigma, St. Louis, Mo.) and 10% fetal calf serum (FCS; Invitrogen, Paisley, UK), and then incubated over night at 4 uC with the primary antibody diluted in PBS/0.1% Triton X-100 plus 1% FCS. After 5 washes with PBS/0.1% Triton X-100, the sections are incubated for 1 h at room temperature with Cy2 conjugated anti-rabbit IgG (H+L) antibody (Jackson lmmunoresearch, Newmarket, UK) or Alexa Fluor 594 donkey anti-goat IgG (H+L) antibody (Invitro-gen), each diluted 1/1,000. Sections are finally washed 5 times in PBS/0.1% Triton X-100 and mounted in glycerol/PBS (1:1).

Cell Death/Survival Quantification

An ELISA, using a combination of antibodies recognizing histones and DNA (Roche Diagnostics), is used to quantify cell death in the retina in vivo. This method quantifies cell death as the level of soluble nucleosomes present in cytosolic retinal extracts. Briefly, retinas are homogenized in 200 ml containing 16 protease inhibitor (Roche Diagnostics) and centrifuged at 20,0006 g for 10 min. A portion of supernatant is used to quantify proteins by standard methods (BioRad Protein Assay), and the rest is diluted 1/15 (illuminated) or 1/10 (darkness) in the supplied buffer and processed as indicated by the manufacturer. Results are shown as optical density (OD) per unit of protein (mg) present in the extract.

To quantify cell survival in vitro, 661W cell cultures are fixed with 4% paraformaldehyde (PFA) (Merck, Darmstadt, Germany) for 15 min at room temperature, and the nuclei are stained with 1 mg/ml bisbenzimide (Sigma). The degree of cell survival is determined by counting the number of non-pyknotic nuclei in the cultures. Cells are counted by using a Leica DM16000 B inverted microscope (Leica) with phase contrast and epifluorescence illumination. Randomly taken pictures are taken with a Leica DFC350 FX digital camera (Leica), and subsequently analyzed with ImageJ (NIH, Bethesda, Md.) software using the particle analysis (nucleus counter) plugin. On average, 8,000 nuclei are analyzed per experimental point.

Blockage of ProNGF Signaling

Anti-sortilin antibodies at different concentrations are injected into one eye of illuminated or control mice to prevent proNGF signaling in vivo. For eye injections, mice are anesthetized with isoflurane. Then, 1 ml of solution containing an anti-sortilin antibody is injected just behind the limbus with a 33 gauge beveled needle (World Precision Instruments, Sarasota, Fla.) using a NanoFil microsyringe (World Precision Instruments). The contralateral eye is injected with 1 ml of the vehicle or an irrelevant IgG used as control. The mice are then maintained in complete darkness for an additional 24 h period prior to analysis.

For in vitro experiments, the anti-sortilin antibody or vehicle (PBS) is added to the cultures at the beginning of intense light treatments.

Alternative Assays

Further assays, including disease models, can be used to assess the beneficial effect of administration of an anti-sortilin antibody under pathological conditions. Alternative assays are, for example, the streptozotocin-induced mouse model as described in Example 2, or the following disease models.

Optic nerve (ON) axotomy is a model of acute degeneration of retinal ganglion cells induced by transection of the optic nerve (Templeton 2012). Proof-of-concept studies are performed in sortilin knockout mice, where acute retinal neurodegeneration is induced by ON axotomy. The end point is 7 or 14 days after transection. Retinal images are acquired by OCT, OPs by ERG, and the number of surviving RGC's will be quantified as described in Example 3. In another experiment, experimental eyes from WT mice are injected intravitreally with anti-sortilin antibodies, whereas the contralateral eye is injected with control antibodies. The injection is performed immediately after ON transection and again at day 7. Retinal images are acquired by OCT, ERG analysis performed, and the number of surviving RGC's will be quantified as described above and in Example 3.

The oxygen-induced retinopathy model (Smith 1994) is a model for vascular pathology in the retina. Sortilin knockout mouse pups are exposed to hyperoxia (75% oxygen) from P7 to P12. This leads to capillary depletion and upon return to room air at day P12 results in retinal ischemia and proliferative vascular disease in the retina. Euthanization of WT and sortilin knockout mice pups at different time points reveals information on mechanisms involved in capillary obliteration (P12) or retinal neovascularization (P17). Eyes are enucleated and investigated by isolectin staining of retinal flatmounts.

Results:

Inhibitory anti-sortilin antibodies, which are able to inhibit apoptosis in vivo and/or in vitro, or who have other beneficial effects in the pathological setting, are identified.

Conclusion:

The identified inhibitory anti-sortilin antibodies can be used in the treatment, prevention, detection and/or diagnosis of a disease or disorder of the retina, the choroid and/or the optic nerve.

### Example 8

### Overview of Sequences

Amino acid sequences as referred to throughout the text are presented here.

SEQ ID NO. 1: Human full-length pre-pro-sortilin (Q99523)
MERPWGAADGLSRWPHGLGLLLLLQLLPPSTLSQDRLDAPPPPAAPLPRWSGPIGVSWGL
RAAAAGGAFPRGGRWRRSAPGEDEECGRVRDFVAKLANNTHQHVFDDLRGSVSLSWVGDS
TGVILVLTTFHVPLVIMTFGQSKLYRSEDYGKNFKDITDLINNTFIRTEFGMAIGPENSG
KVVLTAEVSGGSRGGRIFRSSDFAKNFVQTDLPFHPLTQMMYSPQNSDYLLALSTENGLW
VSKNFGGKWEEIHKAVCLAKWGSDNTIFFTTYANGSCKADLGALELWRTSDLGKSFKTIG
VKIYSFGLGGRFLFASVMADKDTTRRIHVSTDQGDTWSMAQLPSVGQEQFYSILAANDDM
VFMHVDEPGDTGFGTIFTSDDRGIVYSKSLDRHLYTTTGGETDFTNVTSLRGVYITSVLS
EDNSIQTMITFDQGGRWTHLRKPENSECDATAKNKNECSLHIHASYSISQKLNVPMAPLS
EPNAVGIVIAHGSVGDAISVMVPDVYISDDGGYSWTKMLEGPHYYTILDSGGIIVAIEHS
SRPINVIKFSTDEGQCWQTYTFTRDPIYFTGLASEPGARSMNISIWGFTESFLTSQWVSY
TIDFKDILERNCEEKDYTIWLAHSTDPEDYEDGCILGYKEQFLRLRKSSVCQNGRDYVVT
KQPSICLCSLEDFLCDFGYYRPENDSKCVEQPELKGHDLEFCLYGREEHLTTNGYRKIPG
DKCQGGVNPVREVKDLKKKCTSNFLSPEKQNSKSNSVPIILAIVGLMLVTVVAGVLIVKK
YVCGGRFLVHRYSVLQQHAEANGVDGVDALDTASHTNKSGYHDDSDEDLLE
SEQ ID NO. 2: Extracellular domain of sortilin
QDRLDAP PPPAAPLPRW
SGPIGVSWGL RAAAAGGAFP RGGRWRRSAP GEDEECGRVR DFVAKLANNT
HQHVFDDLRG SVSLSWVGDS TGVILVLTTF HVPLVIMTFG QSKLYRSEDY
GKNFKDITDL INNTFIRTEF GMAIGPENSG KVVLTAEVSG GSRGGRIFRS
SDFAKNFVQT DLPFHPLTQM MYSPQNSDYL LALSTENGLW VSKNFGGKWE
EIHKAVCLAK WGSDNTIFFT TYANGSCKAD LGALELWRTS DLGKSFKTIG
VKIYSFGLGG RFLFASVMAD KDTTRRIHVS TDQGDTWSMA QLPSVGQEQF
YSILAANDDM VFMHVDEPGD TGFGTIFTSD DRGIVYSKSL DRHLYTTTGG
ETDFTNVTSL RGVYITSVLS EDNSIQTMIT FDQGGRWTHL RKPENSECDA
TAKNKNECSL HIHASYSISQ KLNVPMAPLS EPNAVGIVIA HGSVGDAISV
MVPDVYISDD GGYSWTKMLE GPHYYTILDS GGIIVAIEHS SRPINVIKFS
TDEGQCWQTY TFTRDPIYFT GLASEPGARS MNISIWGFTE SFLTSQWVSY
TIDFKDILER NCEEKDYTIW LAHSTDPEDY EDGCILGYKE QFLRLRKSSV
CQNGRDYVVT KQPSICLCSL EDFLCDFGYY RPENDSKCVE QPELKGHDLE
FCLYGREEHL TTNGYRKIPG DKCQGGVNPV REVKDLKKKC TSNFLSPEKQ
NSKSNS
SEQ ID NO. 3: Neurotensin binding site 1
SVMAD KDTTRRIHVS TDQGDTWSMA QLPSVGQEQF Y
SEQ ID NO. 4: Neurotensin binding site 2
GSVSL
SEQ ID NO. 5: Binding site for the propeptide of sortilin
SISQ KLNVPMAPLS EPNAVGIVIA HGSVG
SEQ ID NO. 6: proNGF binding site 1
RIFRSSDFAKNE
SEQ ID NO. 7: proNGF binding site 2
SVMAD KDTTRRIHVS TDQGDTWSMA QLPSVGQEQF Y
SEQ ID NO. 8: proNGF binding site 3
SEDNSIQTM
SEQ ID NO. 9: proNGF binding site 4
IHASI
SEQ ID NO. 10: proNGF binding site 5
VIA HGSVGDAIS
SEQ ID NO. 11: proNGF binding site 6
GYRKIPG DKCQGGVNPV
SEQ ID NO. 12: NGF binding site
CEEKDYTIWLAHSTDPEDYEDGCILGYKEQFLRLRKSSVCQNGRDYVVTKQPSICLCSLE
DFLCDFGYYRPENDSKCVEQPELKGHDLEFCLYGREEHLTTNGYRKIPGDKCQGGVNPVR
EVKDLKKKCTSNFLSPEKQNSKSNS
Sequence of the Neurotensin binding site 3:
TGL

### REFERENCES

Ali, T. K., et al., Diabetes-induced peroxynitrite impairs the balance of pro-nerve growth factor and nerve growth factor, and causes neurovascular injury. Diabetologia, 2011. 54(3): p. 657-68.
Antonetti, D. A., R. Klein, and T. W. Gardner, Diabetic retinopathy. N Engl J Med, 2012. 366(13): p. 1227-39.
Askou, A. L., et al., Suppression of Choroidal Neovascularization by AAV-Based Dual-Acting Antiangiogenic Gene Therapy. Mol Ther Nucleic Acids, 2019. 16: p. 38-50.
Askou, A. L., et al., Suppression of Choroidal Neovascularization in Mice by Subretinal Delivery of Multigenic Lentiviral Vectors Encoding Anti-Angiogenic MicroRNAs. Hum Gene Ther Methods, 2017. 28(4): p. 222-233.
Carlo, A. S., et al., The pro-neurotrophin receptor sortilin is a major neuronal apolipoprotein E receptor for catabolism of amyloid-beta peptide in the brain. J Neurosci, 2013. 33(1): p. 358-70.
De Clerck, E. E. B., et al., Macular thinning in prediabetes or type 2 diabetes without diabetic retinopathy: the Maastricht Study. Acta Ophthalmol, 2018. 96(2): p. 174-182.
Evans, S. F., et al., Neuronal brain-derived neurotrophic factor is synthesized in excess, with levels regulated by sortilin-mediated trafficking and lysosomal degradation. J Biol Chem, 2011. 286(34): p. 29556-67.
Finan, G. M., H. Okada, and T. W. Kim, BACE1 retrograde trafficking is uniquely regulated by the cytoplasmic domain of sortilin. J Biol Chem, 2011. 286(14): p. 12602-16.
Glerup, S, et al., SorCS2 regulates dopaminergic wiring and is processed into an apoptotic two-chain receptor in peripheral glia. Neuron 2014, June 4; 82(5):1074-87.
Gustafsen, C., et al., Sortilin and SorLA display distinct roles in processing and trafficking of amyloid precursor protein. J Neurosci, 2013. 33(1): p. 64-71.
Habault J., et al., Recent Advances in Cell Penetrating Peptide-Based Anticancer Therapies. Molecules, 2019 Mar. 7; 24(5).
Hermey, G., et al., Characterization of sorCS1, an alternatively spliced receptor with completely different cytoplasmic domains that mediate different trafficking in cells. J Biol Chem, 2003. 278(9): p. 7390-6.
Henderson, R. H., Inherited retinal dystrophies. Paediatrics and Child Health, Volume 30, Issue 1, January 2020, Pages 19-27.
Jacobsen, L., et al., Activation and functional characterization of the mosaic receptor SorLA/LR11. J Biol Chem, 2001. 276(25): p. 22788-96.
Jansen, P., et al., Roles for the pro-neurotrophin receptor sortilin in neuronal development, aging and brain injury. Nat Neurosci, 2007. 10(11): p. 1449-57.
Kjolby, M., et al., Sort1, encoded by the cardiovascular risk locus 1p13.3, is a regulator of hepatic lipoprotein export. Cell Metab, 2010. 12(3): p. 213-23.
Mohamed, R. et al., Imbalance of the Nerve Growth Factor and Its Precursor: Implication in Diabetic Retinopathy. J Clin Exp Ophthalmol. 2015 October; 6(5). pii: 483.
Nykjaer, A., et al., Sortilin is essential for proNGF-induced neuronal cell death. Nature, 2004. 427(6977): p. 843-8.
Oshitari, T., K. Hanawa, and E. Adachi-Usami, Changes of macular and RNFL thicknesses measured by Stratus OCT in patients with early stage diabetes. Eye (Lond), 2009. 23(4): p. 884-9.
Petersen, C. M., et al., Molecular identification of a novel candidate sorting receptor purified from human brain by receptor-associated protein affinity chromatography. J Biol Chem, 1997. 272(6): p. 3599-605.
Pincelli Netto, M., et al., Macular Inner Retinal Layer Thinning in Diabetic Patients without Retinopathy Measured by Spectral Domain Optical Coherence Tomography. Med Hypothesis Discov lnnov Ophthalmol, 2018. 7(3): p. 133-139.
Ruiz-Ederra, J. et al., Mouse model of sustained elevation in intraocular pressure produced by episcleral vein occlusion. Exp Eye Res, 2006. 82(5): p. 879-84.
Santos, A. M., et al., Sortilin participates in light-dependent photoreceptor degeneration in vivo. PLoS One, 2012; 7(4):e36243.
Skeldal, S., et al., Proteolytic processing of the p75 neurotrophin receptor: A prerequisite for signalling?: Neuronal life, growth and death signalling are crucially regulated by intra-membrane proteolysis and trafficking of p75(NTR). Bioessays, 2011. 33(8): p. 614-25.
Smith, L. E., et al., Oxygen-induced retinopathy in the mouse. Invest Ophthalmol Vis Sci, 1994. 35(1): p. 101-11
Templeton, J. P. and E. E. Geisert, A practical approach to optic nerve crush in the mouse. Mol Vis, 2012. 18: p. 2147-52.)
Wong, T. Y., et al., Diabetic retinopathy. Nat Rev Dis Primers, 2016. 2: p. 16012. NCD Risk Factor Collaboration (NCD-RisC). Worldwide trends in diabetes since 1980: a pooled analysis of 751 population-based studies with 4.4 million participants (vol 387, pg 1513, 2016). Lancet, 2017. 389(10068): p. E2-E2.
Yang, M., et al., Precursor of brain-derived neurotrophic factor (proBDNF) forms a complex with Huntingtin-associated protein-1 (HAP1) and sortilin that modulates proBDNF trafficking, degradation, and processing. J Biol Chem, 2011. 286(18): p. 16272-84.
Yau, J. W. Y., et al., Global Prevalence and Major Risk Factors of Diabetic Retinopathy. Diabetes Care, 2012. 35(3): p. 556-564.

1. A composition comprising or consisting of an anti-sortilin antibody or an antigen binding fragment thereof, for use in the treatment or prevention of a disease or disorder of the retina, the choroid and/or the optic nerve.

2. The composition for use according to any one of the preceding claims, wherein the antibody or antigen binding fragment thereof is a polyclonal antibody.

3. The composition for use according to any one of the preceding claims, wherein the antibody or antigen binding fragment thereof is a monoclonal antibody.

4. The composition for use according to any one of the preceding claims, wherein the antibody or antigen binding fragment thereof is a human anti-sortilin antibody.

5. The composition for use according to any one of the preceding claims, wherein the antibody or antigen binding fragment thereof is a goat anti-sortilin antibody or a rabbit anti-sortilin antibody.

6. The composition for use according to any one of the preceding claims, wherein the antibody or antigen binding fragment thereof is an IgG antibody.

7. The composition for use according to any one of the preceding claims, wherein the antibody or antigen binding fragment thereof binds to at least one amino acid of the amino acid sequence of SEQ ID NO. 1.

8. The composition for use according to any one of the preceding claims, wherein the antibody or antigen binding fragment thereof binds to at least one amino acid of the amino acid sequence of SEQ ID NO. 2.

9. The composition for use according to any one of the preceding claims, wherein the antibody or antigen binding fragment thereof binds to at least one amino acid of the amino acid sequence of SEQ ID NO. 3.

10. The composition for use according to any one of the preceding claims, wherein the antibody or antigen binding fragment thereof binds to at least one amino acid of the amino acid sequence of SEQ ID NO. 4.

11. The composition for use according to any one of the preceding claims, wherein the antibody or antigen binding fragment thereof binds to at least one amino acid of the amino acid sequence TGL, wherein said amino acid sequence is comprised in the Neurotensin binding site 3.

12. The composition for use according to any one of the preceding claims, wherein the antibody or antigen binding fragment thereof binds to at least one amino acid of the amino acid sequence of SEQ ID NO. 5.

13. The composition for use according to any one of the preceding claims, wherein the antibody or antigen binding fragment thereof binds to at least one amino acid of the amino acid sequence of SEQ ID NO. 6.

14. The composition for use according to any one of the preceding claims, wherein the antibody or antigen binding fragment thereof binds to at least one amino acid of the amino acid sequence of SEQ ID NO. 7.

15. The composition for use according to any one of the preceding claims, wherein the antibody or antigen binding fragment thereof binds to at least one amino acid of the amino acid sequence of SEQ ID NO. 8.

16. The composition for use according to any one of the preceding claims, wherein the antibody or antigen binding fragment thereof binds to at least one amino acid of the amino acid sequence of SEQ ID NO. 9.

17. The composition for use according to any one of the preceding claims, wherein the antibody or antigen binding fragment thereof binds to at least one amino acid of the amino acid sequence of SEQ ID NO. 10.

18. The composition for use according to any one of the preceding claims, wherein the antibody or antigen binding fragment thereof binds to at least one amino acid of the amino acid sequence of SEQ ID NO. 11.

19. The composition for use according to any one of the preceding claims, wherein the antibody or antigen binding fragment thereof binds to at least one amino acid of the amino acid sequence of SEQ ID NO. 12.

20. The composition for use according to any one of the preceding claims, wherein the antibody or antigen-binding fragment thereof is selected from the group consisting of Fab, F(ab′)₂, Fv fragments, Fab-like fragments, single variable domains, and nanobodies.

21. The composition for use according to any of the preceding claims, wherein the antibody or antigen binding fragment thereof is humanised.

22. The composition for use, according to any one of the preceding claims, wherein the antibody or antigen binding fragment thereof is conjugated to a cell-penetrating peptide such as TAT-peptide.

23. The composition for use according to any one of the preceding claims, wherein the disease is a disease of the retina.

24. The composition for use according to any one of the preceding claims, wherein the disease of the retina is selected from the group consisting of diabetic retinopathy, retinal vein occlusion, retinal artery occlusion, retinopathy of prematurity, Coats' disease, tapetoretinal degeneration, hereditary retinal dystrophy or any combination thereof.

25. The composition for use according to any one of the preceding claims, wherein the disease is a disease of the choroid.

26. The composition for use according to any one of the preceding claims, wherein the disease of the choroid is selected from the group consisting of age-related macular degeneration, choroiditis or any combination thereof.

27. The composition for use according to any one of the preceding claims, wherein the disease is a disease of the optic nerve.

28. The composition for use according to any one of the preceding claims, wherein the disease of the optic nerve is selected from the group consisting of glaucoma, autosomal dominant optic neuropathy, Leber's hereditary optic neuropathy, optic neuritis, anterior ischemic optic neuropathy, toxic diseases of the optic nerve and nerve head or any combination thereof.

29. The composition for use according to any one of the preceding claims, wherein the disease or disorder is retinal degeneration or retinal tissue loss.

30. The composition for use, according to any one of the preceding claims, wherein the composition is for use in the treatment of a mammal.

31. The composition for use, according to any one of the preceding claims, wherein the composition is for use in the treatment of a human.

32. The composition for use, according to any one of the preceding claims, wherein the antibody or antigen binding fragment thereof is administered intravitreally.

33. The composition for use, according to any one of the preceding claims, wherein the antibody or antigen binding fragment thereof is administered in the form of eye drops.

34. The composition for use, according to any one of the preceding claims, wherein the antibody or antigen binding fragment thereof is administered topically on the eye.

35. The composition for use, according to any one of the preceding claims, wherein the antibody or antigen binding fragment thereof is administered in the form of a gel.

36. Use of a composition comprising or consisting of an anti-sortilin antibody or an antigen binding fragment thereof, for the manufacture of a medicament for the treatment or prevention of a disease of the retina, the choroid and/or the optic nerve.

37. A method of treatment of a disease of the retina, the choroid and/or the optic nerve, the method comprising administering to an individual in need thereof a therapeutically effective amount of an anti-sortilin antibody or an antigen binding fragment thereof.

38. A composition comprising or consisting of an anti-sortilin antibody or an antigen binding fragment thereof, for use in the diagnosis of a disease of the retina, the choroid and/or the optic nerve.

39. A method for detecting a disease of the retina, the choroid and/or the optic nerve by using an anti-sortilin antibody or an antigen binding fragment thereof, said method comprising analysing in a sample obtained from a mammalian retina, choroid or optic nerve, the presence or absence of an antigen comprising a sortilin polypeptide, wherein the presence of the antigen is indicative of a disease of the retina, the choroid and/or the optic nerve.
